(19) United States
(12) Patent Application Publication
(10) Pub. No.: US 2019/0232034 A1
(43) Pub. Date: Aug. 1, 2019

(54) METHOD OF MANUFACTURING MICRONEEDLE BY USING VIBRATION AND GRAVITY

(71) Applicant: Sanghyuk Lee, Gyeonggi-do (KR)

(72) Inventor: Sanghyuk Lee, Gyeonggi-Do (KR)

(21) Appl. No.: 16/337,999

(22) PCT Filed: Aug. 22, 2017
(86) PCT No.: PCT/KR2017/009158

§ 371 Date: Mar. 29, 2019

(30) Foreign Application Priority Data
Sep. 30, 2016 (KR) 10-2016-0126265

(51) Int. Cl. A61M 37/00; B29C 67/00; B29C 69/00

(52) U.S. Cl. CPC A61M 37/0015; B29C 67/00; B29L 2031/7544; A61M 2037/0023; A61M 2037/0053; B29C 69/001

(57) ABSTRACT

A method of manufacturing a microneedle and a structure with an array of microneedles manufactured by using the method, in which a size, height, and shape of the microneedle formed on a base may be adjusted. The multiple microneedles may be uniform in size, height, and shape even though the multiple microneedles are simultaneously formed on the base. The method is economical because precise control is not required and an area of the structure with the array of microneedles may be increased only with a simple facility. The structure with an array of multiple fine microneedles formed on the base (or substrate), includes the microneedle having an upper end having a fine diameter that does not cause pain or trauma to the skin, and includes sufficient hardness required to penetrate a skin layer, and sufficient effective length and a stable shape delivering active ingredients to capillaries.

### CROSS-REFERENCE TO RELATED APPLICATIONS

This application is the U.S. national stage of PCT/KR2017/009158 filed Aug. 22, 2017, which claims priority of Korean Patent Application No. 10-2016-0126265 filed on Sep. 30, 2016 which are incorporated herein by reference in their entirety.

### FIELD OF THE INVENTION

The present invention relates to a method of manufacturing a microneedle by using vibration and gravity.

### BACKGROUND OF THE INVENTION

A method of improving efficiency in delivering various types of active ingredients, such as medicines or bioactive substances, for cosmetic or medical purpose, into a human body has always been an important issue. In case of oral administration, various types of active ingredients such as medicines or bioactive substances may digest in a gastrointestinal tract and may disappear due to a mechanism in a liver, and as a result, it may be difficult to obtain an effect of delivering a desired and sufficient amount of ingredients. In addition, a method of inputting active ingredients directly into a human body through skin by using an injection needle may solve the above-mentioned problem occurring in case of oral administration but has caused problems of pain at injection sites and local injuries to skin.

Recently, to solve the above-mentioned problems, there has been developed a method of inputting active ingredient into the skin by using a microneedle that has a finer size than the injection needle in the related art. Since the microneedle has a much finer size than the injection needle in the related art, the microneedle may reduce pain caused by the penetration of the microneedle into the skin and reduce skin trauma in comparison with the injection needle in the related art.

In general, it has been known that the human's skin includes a horny layer (<20 m), an epidermal layer (<100 μm), and a dermal layer (300 to 2,500 μm) in this order. A diameter of an upper end of the microneedle needs to be sufficiently fine and a length of the microneedle needs to be sufficiently long in order to effectively deliver various types of active ingredients, such as medicines and bioactive substances, into the human body without causing pain in the respective skin layers. In addition, the microneedle needs to have a stable shape so as not to be easily broken at the time of applying the microneedle directly to the skin. The upper end of the microneedle needs to have sufficient hardness so as to smoothly penetrate the horny layer of the skin so that active ingredients such as medicines may be sufficiently delivered into the skin.

Meanwhile, continuous and various researches have been carried out on a method of manufacturing a microstructure in which the microneedles are uniformly arranged on a base or substrate and the microneedles meet the requirements related to the fine diameter of the upper end, the sufficient length and hardness, and the stable shape. For example, Korean Patent No. 10-1254240 proposed a method of manufacturing a microstructure by bringing two substrates into contact with each other with a viscous composition interposed therebetween, moving the substrates relative to each other to extend the viscous composition between the substrate, and then coagulating the viscous composition. Korean Patent No. 10-1590172 proposed a method of manufacturing a microstructure by exerting centrifugal force on a viscous composition to extend the viscous composition.

However, in the case of Korean Patent No. 10-1254240, the upper and lower substrates need to be moved relative to each other in order to manufacture the microstructure, and it is necessary to very precisely control the relative movements of the substrates. In particular, if a spacing distance between the upper and lower substrates is excessively small during a process of decreasing the distance between the upper and lower substrates, the viscous composition is spread on the substrates and thus cannot be extended. If the spacing distance is not sufficiently small, the viscous composition is disconnected or becomes imbalanced at the time of extending the viscous composition. Therefore, superprecision parallel control is required for the relative movements of the upper and lower substrates, but the superprecision parallel control takes an excessively large amount of process time and incurs a large amount of costs, which causes a fatal disadvantage in terms of mass-production.

In addition, in the case of Korean Patent No. 10-1590172, the centrifugal force is exerted, only in one direction, on the viscous composition at the time of manufacturing the microstructure. As a result, there is a problem in that the manufactured microneedle has an asymmetrical shape or a non-uniform size. There is also a problem in that it is difficult to manufacture the microstructure having a large area because of the nature of the manufacturing method that attaches the substrate to a rotary arm of a centrifugal separator and rotates the substrate.

### SUMMARY OF THE INVENTION

The present invention has been made in an effort to provide a method of manufacturing a microneedle and a structure with an array of microneedles manufactured by using the method, in which a size, a height, and/or a shape of the microneedle formed on a base may be easily and optionally adjusted and the multiple microneedles may be uniform in size, height, and/or shape even though the multiple microneedles are simultaneously formed on the base, such that it is possible to manufacture the structure with an array of multiple fine microneedles formed on the base (or substrate), in which the microneedle has an upper end having a fine diameter that does not cause pain or trauma to skin, the microneedle has sufficient hardness required when the microneedle penetrates a skin layer, and the microneedle has a sufficient effective length and a stable shape that may allow effective and active ingredients to be effectively delivered to capillaries.

An exemplary embodiment of the present invention provides a method of manufacturing a microneedle, the method including: a) dropping a viscous composition at one or more points on an upper surface of a first base; b) reversing the first base so that the surface on which the viscous composition is positioned is positioned at a lower side of the first base and faces an upper surface of a second base spaced apart from the first base at a predetermined interval; c) forming a viscous composition column, which is in contact with the first base and the second base, from the viscous composition attached to the first base by vibrating the first base or both of the first base and the second base to allow the viscous composition to extend downward vertically by vibration and gravity; and d) drying and cutting the formed viscous composition column, in which a size or height of the microneedle to be formed is adjusted by controlling an interval between the first base and the second base in step b.

Another exemplary embodiment of the present invention provides a structure with an array of microneedles which is manufactured by the above-mentioned method and includes one or more microneedles formed on the base.

According to the method of manufacturing a microneedle according to the present invention, it is possible to manufacture the structure with the array of multiple fine microneedles formed on the base (or substrate), in which the microneedle has the upper end having a fine diameter that does not cause pain or trauma to skin, the microneedle has sufficient hardness required when the microneedle penetrates a skin layer, and the microneedle has a sufficient effective length and a stable shape that may allow effective and active ingredients to be effectively delivered to capillaries.

In addition, according to the manufacturing method according to the present invention, it is possible to easily and optionally adjust sizes, heights, and shapes of the multiple microneedles formed on the base and to make the multiple microneedles uniform in size, height, and shape at the time of simultaneously forming the multiple microneedles on the base.

In addition, the manufacturing method according to the present invention is economical because precise control is not required and an area of the structure with the array of microneedles may be increased only with a simple facility.

### BRIEF DESCRIPTION OF THE DRAWINGS

FIG. 1 is a view schematically illustrating a method of manufacturing a microneedle according to an exemplary embodiment of the present invention.

FIG. 2 is a photograph illustrating a process of manufacturing a microneedle in accordance with the method of manufacturing the microneedle according to the exemplary embodiment of the present invention.

FIG. 3 is a view illustrating a state in which one or more microneedles are formed on a base in a structure with an array of microneedles manufactured according to the exemplary embodiment of the present invention.

FIG. 4 is a view schematically illustrating a transdermal patch structure for delivering medicines by using the structure with the array of microneedles manufactured according to the exemplary embodiment of the present invention.

FIG. 5 is a photograph illustrating a state in which a microneedle is not effectively manufactured when the microneedle is manufactured in the related art.

FIG. 6 is a photograph illustrating a state in which a microneedle is not effectively manufactured when the microneedle is manufactured in the related art.

### DETAILED DESCRIPTION OF THE PREFERRED EMBODIMENTS

Hereinafter, an exemplary embodiment of the present invention will be described in detail with reference to the accompanying drawings. Detailed descriptions of publicly known functions and configurations will be omitted when it is determined that the detailed descriptions may unnecessarily obscure the subject matter of the present invention. Further, the following descriptions are provided for explaining the exemplary embodiment of the present invention, and the present invention is not limited thereto.

Meanwhile, it should be appreciated that the terms ‘first base’ and ‘second base’ used throughout the claims of the present invention and the specification are arbitrarily provided to conveniently distinguish the bases on the order thereof.

Method of Manufacturing Microstructure

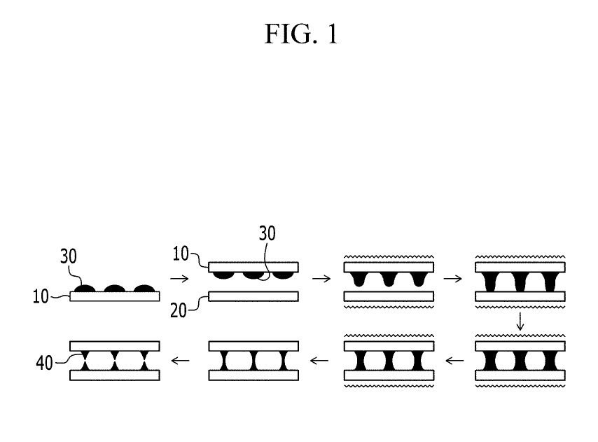

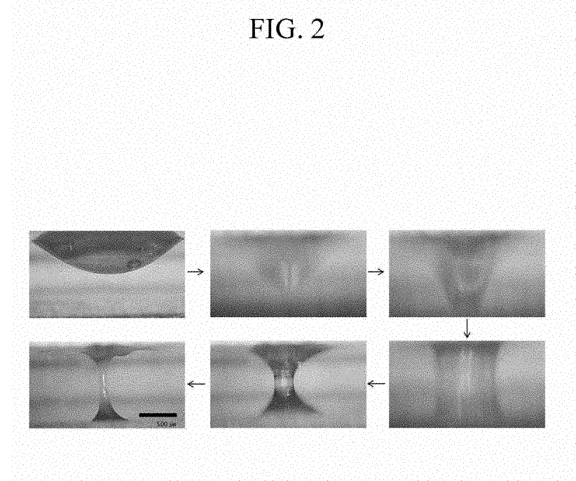

A method of manufacturing a microneedle according to an exemplary embodiment of the present invention includes: a) dropping a viscous composition 30 at one or more points on an upper surface of a first base 10; b) reversing the first base so that the surface on which the viscous composition is positioned, is positioned at a lower side of the first base and faces an upper surface of a second base 20 spaced apart from the first base at a predetermined interval; c) forming a viscous composition column, which is in contact with the first base and the second base, from the viscous composition attached to the first base by vibrating the first base or both of the first base and the second base to allow the viscous composition to extend downward vertically by vibration and gravity; and d) drying and cutting the formed viscous composition column (see FIGS. 1 and 2).

First, the viscous composition is dropped at one or more points on the upper surface of the first base (step a).

Meanwhile, the first base may have a film or sheet structure including a polymeric resin-based material, and the first base per se may be rigid or flexible. In addition, the base may have a through hole as necessary or may be structured to deliver medicine to the skin through the through hole from an opposite surface of the microneedle.

In the exemplary embodiment of the present invention, the base may have a film or sheet structure including one or more polymeric resin-based materials selected from a group consisting of polyethylene, polypropylene, polyvinyl chloride resin, polyethylene terephthalate (PET), nylon, epoxy, polyimide, polyester, urethane, acrylic, polycarbonate, urea, melanin, rubber chloride, polyvinyl alcohol, polyvinyl ester, vinylidenefluoride-hexafluoropropylene copolymer (PVDF-co-HFP), polyvinylidenefluoride (PVDF), polyacrylonitrile, polymethylmethacrylate, polytetrafluoroethylene (PTFE), stylenebutadiene rubber (SBR), and ethylene-propylene-diene copolymer (EPDM).

Meanwhile, the viscous composition is used to manufacture the microneedle through a series of processes to be described below and may be a material having biocompatibility and biodegradability if the microneedle is used for medical purpose.

Here, the biocompatible material means a chemically inert material which is not toxic to a human body. Further, the biodegradable material means a material that may be decomposed by a bodily fluid, an enzyme, or a microorganism in a body. According to the exemplary embodiment of the present invention, the viscous composition may include one or more materials selected from a group consisting of hydroxypropylmethyl cellulose, hydroxyalkyl cellulose, ethylhydroxyethyl cellulose, alkyl cellulose, and carboxymethyl cellulose. In addition, according to the exemplary embodiment of the present invention, the viscous composition may include functional materials, for example, chemical medicines, protein medicines, peptide medicines, nucleic acid molecules for gene therapy, and nanoparticle cosmetic ingredients (e.g., an anti-wrinkle agent, a skin aging inhibitor, and a skin whitening agent) that penetrate into the skin and perform particular medicinal or cosmetic functions.

In addition, in the exemplary embodiment of the present invention, the viscous composition may include one or more biocompatible materials selected from a group consisting of chitosan, collagen, gelatin, hyaluronic acid (HA), alginic acid, pectin, carrageenan, chondroitin (sulfate), dextran (sulfate), polylysine, carboxymethyl chitin, fibrin, agarose, pullulan, and cellulose that are biodegradable or soluble in biological tissues when the material is injected into the skin.

In addition, in the exemplary embodiment of the present invention, the viscous composition may include hyaluronic acid of 5 to 50 kDa of 10 to 80% w/v, particularly, hyaluronic acid of 15 to 40 kDa of 30 to 50% w/v, more particularly, hyaluronic acid of 29 kDa of 40% w/v.

Meanwhile, according to the exemplary embodiment of the present invention, in step a, it is possible to adjust a size of the microneedle finally formed on the base by controlling the amount of viscous composition to be dropped on the upper surface of the first base. In addition, the viscous composition may be dropped at the multiple points on the upper surface of the first base in accordance with the number of microneedles to be formed on the base. In this case, the viscous composition may be dropped on the base so as to have or not to have regularity in accordance with the intended arrangement shape of the microneedles.

Next, the first base is reversed so that the surface on which the viscous composition is positioned, is positioned at the lower side of the first base and faces the upper surface of the second base spaced apart from the first base at a predetermined interval (step b).

Meanwhile, the second base may be identical or different in material or shape to/from the above-mentioned first base. However, in the case in which the second base is different in material and shape from the first base, the material and the shape of the second base may be selected as necessary from the ranges of the material and shape of the first base.

Meanwhile, according to the exemplary embodiment of the present invention, the size (or height) of the microneedle to be formed may be adjusted by controlling an interval between the first base and the second base in step b.

That is, in the case of configuring the arrangement in step b, the interval between the first base and the second base is controlled in accordance with a target size, a target height, and a target shape of the microneedle. The reason is that the microneedles are formed on the first base and the second base, respectively, as a middle portion of one viscous composition column formed between the first base and the second base is cut. Therefore, the viscous composition column may have a height which is about 2 times the target size of the microneedle. According to the exemplary embodiment of the present invention, the interval between the first base and the second base may be more than 0 μm and equal to or less than 4,000 μm in step b.

Next, the viscous composition column, which is in contact with the first base and the second base, is formed from the viscous composition attached to the first base by vibrating the first base or both of the first base and the second base to allow the viscous composition to extend downward vertically by vibration and gravity (step c).

According to the exemplary embodiment of the present invention, only the first base may be vibrated or both of the first base and the second base may be simultaneously vibrated in step c.

The vibration may be applied in an up-down direction or a left-right direction or applied complexly in both of the up-down direction and the left-right direction. Meanwhile, when the vibration is applied to the base as described above, the viscous composition attached to the first base is extended downward vertically by vibration and gravity and adhesively attached to the upper surface of the second base. In this case, the viscous composition is not disconnected because of its inherent viscosity, and the viscous composition is formed in the form of a column in a state in which the viscous composition is connected to the first base and the second base.

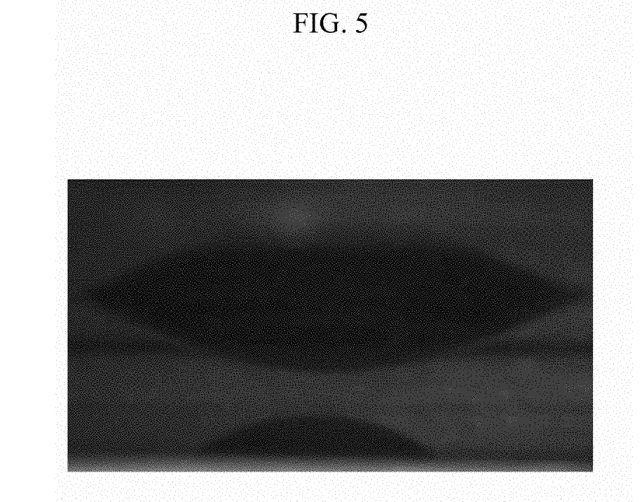

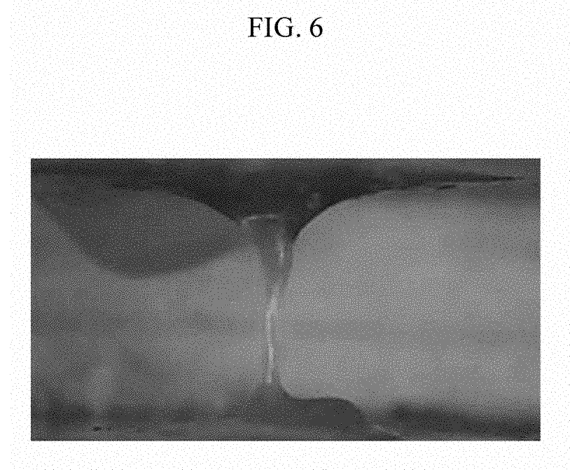

However, if the vibration is not consistently applied even though the viscous composition column has been formed by the initial vibration in step c, the viscous material cannot smoothly flow from the first base to the second base, and as a result, there is a problem in that the column is not maintained but separated at the middle thereof (see FIG. 5), or a viscous composition column having an asymmetric shape, which is not horizontally symmetrical, may be formed (see FIG. 6). Therefore, in step c, a process of consistently applying the vibration may be performed until the viscous composition column has horizontal symmetry and an appropriate shape. When the vibration is consistently applied as described above, the unstable viscous composition is changed to the stable viscous composition by vibration and gravity at the time of forming the viscous composition column.

Meanwhile, in the exemplary embodiment of the present invention, in c step, a shape of the microneedle to be formed may be adjusted by controlling one or more factors selected between vibration time and vibration intensity.

Meanwhile, a device for applying vibration is not particularly limited as long as the device may vibrate the base in the up-down direction, the left-right direction, or both of the up-down direction and the left-right direction. For example, magnetic vibrator may be used as the device.

Next, the formed viscous composition column is dried and cut (step d).

The drying process may be performed as a natural drying process. However, in the exemplary embodiment of the present invention, a bottom of the base is heated so that the viscous composition column is dried together with the bottom of the base, such that the drying time may be shortened. When the drying time is shortened through the above-mentioned process, a degree to which the viscous composition is spread is decreased toward the upper end of the column, and as a result, it is possible to maintain the shape of the viscous composition column that becomes thicker toward a lower side thereof from an upper side thereof. Therefore, it is possible to ensure stability of the microneedle, and as a result, it is possible to effectively ensure hardness of the microneedle.

In this step, the one or more microneedles are formed on the base, the microneedles each having the upper end having a cutting edge shape which is formed by cutting a middle portion of the viscous composition column. A structure with an array of multiple microneedles, which are regularly or irregularly arranged on the base, is manufactured. In this case, the cutting and separating method is not particularly limited, and for example, a separating method using a laser may be performed.

Structure with Array of Microneedles

The structure with the array of microneedles according to the exemplary embodiment of the present invention, which is manufactured by the above-mentioned method of manufacturing a microneedle, includes one or more microneedles 40 formed on the base 10 or 20 (see FIGS. 1 and 2).

According to present invention, the number of one or more microneedles formed on the base may be variously adjusted in accordance with the purpose and intention, and a shape in which the multiple microneedles are arranged may also be adjusted as necessary. For example, the one or more microneedles may be arranged regularly or irregularly arranged on the base.

Meanwhile, according to the present invention, sizes, heights, and shapes of the multiple microneedles formed on the base are easily and optionally adjusted, and the multiple microneedles are easily and uniformly manufactured.

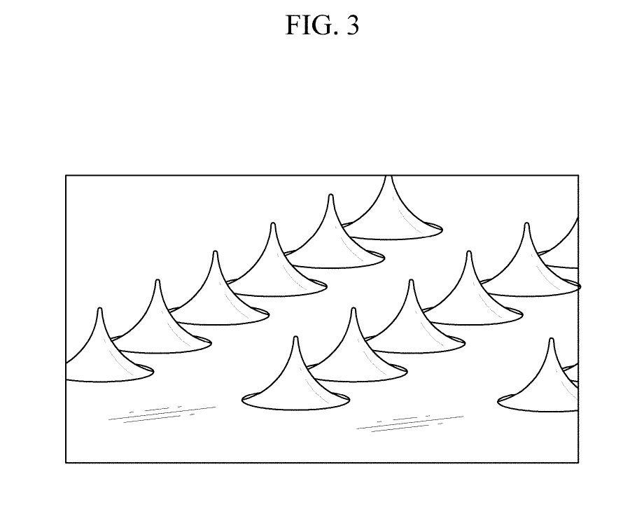

In the above-mentioned structure with the array of microneedles manufactured according to the present invention, the microneedle may have a size (or height) of, for example, 0.1 to 0.4 mm, particularly, 0.25 mm. However, the size or height of the microneedle may vary in accordance with the purpose (see FIG. 3).

Meanwhile, the microneedle, which is formed on the base of the structure with the array of microneedles according to the exemplary embodiment of the present invention, has a lower surface which is in contact with the base, and an upper portion including a cutting edge which is distant from the base. The lower surface may have a cross-sectional diameter which ranges from 50 to 300 μm and is parallel to the base, and the upper portion including a cutting edge may have a cross-sectional diameter which ranges from 5 to 50 μm and is parallel to the base. In particular, the microneedle has sufficient hardness, which is required when the microneedle penetrates the skin layer, while having the fine diameter of the upper end (cutting edge) within the above-mentioned range that does not cause pain or trauma to skin. Further, the microneedle has a sufficient effective length that may allow effective and active ingredients to be effectively delivered to capillaries. Meanwhile, the term ‘cutting edge’ used in the specification and the claims of the present invention is used as meaning that the upper end of the microneedle has a pointed shape (see FIG. 3).

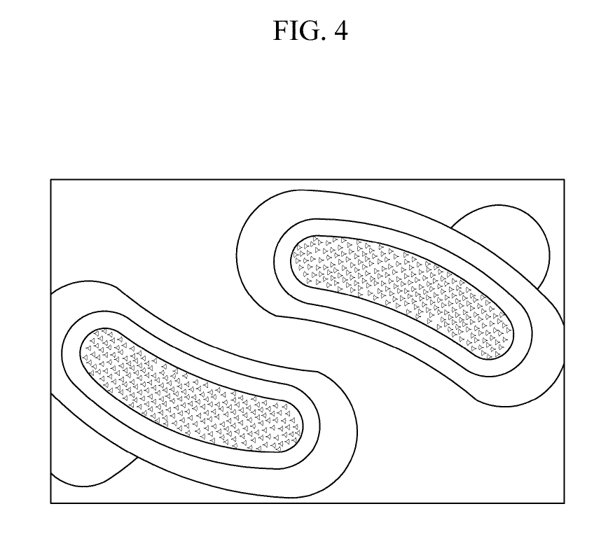

Meanwhile, the above-mentioned structure with the array of microneedles may be utilized in various fields, and particularly, may be utilized for skin care and medical purposes. In the exemplary embodiment of the present invention, the structure with the array of microneedles may be utilized as a transdermal patch for delivering medicines (see FIG. 4).

As described above, according to the method of manufacturing a microneedle according to the present invention, it is possible to manufacture the structure with the array of multiple fine microneedles formed on the base (or substrate), in which the microneedle has the upper end having a fine diameter that does not cause pain or trauma to skin, the microneedle has sufficient hardness required when the microneedle penetrates a skin layer, and the microneedle has a sufficient effective length and a stable shape that may allow effective and active ingredients to be effectively delivered to capillaries.

In addition, according to the manufacturing method according to the present invention, it is possible to easily and optionally adjust sizes, heights, and shapes of the multiple microneedles formed on the base and to make the multiple microneedles uniform in size, height, and shape at the time of simultaneously forming the multiple microneedles on the base.

In addition, the manufacturing method according to the present invention is economical because precise control is not required and an area of the structure with the array of microneedles may be increased only with a simple facility.

### DESCRIPTION OF REFERENCE NUMERALS

10: First base
20: Second base
30: Viscous composition
40: Microneedle

According to the method of manufacturing a microneedle according to the present invention, it is possible to manufacture the structure with the array of multiple fine microneedles formed on the base (or substrate), in which the microneedle has the upper end having a fine diameter that does not cause pain or trauma to skin, the microneedle has sufficient hardness required when the microneedle penetrates a skin layer, and the microneedle has a sufficient effective length and a stable shape that may allow effective and active ingredients to be effectively delivered to capillaries.

According to the manufacturing method according to the present invention, it is possible to easily and optionally adjust sizes, heights, and shapes of the multiple microneedles formed on the base and to make the multiple microneedles uniform in size, height, and shape at the time of simultaneously forming the multiple microneedles on the base.

In addition, the manufacturing method according to the present invention is economical because precise control is not required and an area of the structure with the array of microneedles may be increased only with a simple facility.

1. A method of manufacturing a microneedle, the method comprising the steps of:
a) dropping a viscous composition at one or more points on an upper surface of a first base;
b) reversing the first base so that the surface on which the viscous composition is positioned, is positioned at a lower side of the first base and faces an upper surface of a second base spaced apart from the first base at a predetermined interval;
c) forming a viscous composition column, which is in contact with the first base and the second base, from the viscous composition attached to the first base by vibrating the first base or both of the first base and the second base to allow the viscous composition to extend downward vertically by vibration and gravity; and
d) drying and cutting the formed viscous composition column,
wherein a size or height of the microneedle to be formed is adjusted by controlling an interval between the first base and the second base in step b.

2. The method of claim 1, wherein:
one or both of the first base and the second base have a film or sheet structure including a polymeric resin-based material.

3. The method of claim 1, wherein:
one or both of the first base and the second base have a film or sheet structure including one or more polymeric resin-based materials selected from a group consisting of polyethylene, polypropylene, polyvinyl chloride resin, polyethylene terephthalate (PET), nylon, epoxy, polyimide, polyester, urethane, acrylic, polycarbonate, urea, melanin, rubber chloride, polyvinyl alcohol, polyvinyl ester, vinylidenefluoride-hexafluoropropylene copolymer (PVDF-co-HFP), polyvinylidenefluoride (PVDF), polyacrylonitrile, polymethylmethacrylate, polytetrafluoroethylene (PTFE), stylenebutadiene rubber (SBR), and ethylene-propylene-diene copolymer (EPDM).

4. The method of claim 1, wherein:
the viscous composition is a material having biocompatibility and biodegradability.

5. The method of claim 1, wherein:
the viscous composition includes one or more biocompatible materials selected from a group consisting of chitosan, collagen, gelatin, hyaluronic acid (HA), alginic acid, pectin, carrageenan, chondroitin (sulfate), dextran (sulfate), polylysine, carboxymethyl chitin, fibrin, agarose, pullulan, and cellulose that are biodegradable or soluble in biological tissues when the material is injected into the skin.

6. The method of claim 1, wherein:
the viscous composition includes hyaluronic acid of 5 to 50 kDa of 10 to 80% w/v.

7. The method of claim 1, wherein:
a size of the microneedle to be formed is adjusted by controlling the amount of viscous composition on the upper surface of the first base in step a.

8. The method of claim 1, wherein:
a shape of the microneedle to be formed is adjusted by controlling one or more factors selected between vibration time and vibration intensity in step c.

9. The method of claim 1, further comprising:
heating a bottom of the base when drying the viscous composition column in step d.

10. A structure of an array of microneedles which is manufactured by the method according to claim 1 and includes one or more microneedles formed on a base.

11. The structure of claim 10, wherein:
the one or more microneedles formed on the base are arranged regularly or irregularly on the base.

12. The structure of claim 10, wherein:
the one or more microneedles are uniform in size, height, and shape.

13. The structure of claim 10, wherein:
the microneedle has a lower surface which is in contact with the base, and an upper portion including a cutting edge which is distant from the base, the lower surface has a cross-sectional diameter which ranges from 50 to 300 μm and is parallel to the base, and the upper portion including a cutting edge has a cross-sectional diameter which ranges from 5 to 50 μm and is parallel to the base.

14. The structure of claim 10, which is a transdermal patch for delivering medicine.
